(19) United States
(12) Patent Application Publication
(10) Pub. No.: US 2016/0354516 A1
(43) Pub. Date: Dec. 8, 2016

(54) Minimally Invasive Method of Augmenting a Glans Penis with Intradermal Filler Material

(71) Applicant: Gary Chuang, Hermosa Beach, CA (US)

(72) Inventor: Gary Chuang, Hermosa Beach, CA (US)

(21) Appl. No.: 15/173,701

(22) Filed: Jun. 5, 2016

Related U.S. Application Data
(60) Provisional application No. 62/171,374, filed on Jun. 5, 2015.

(51) Int. Cl. A61L 27/20; A61B 17/34; A61L 27/02; A61F 2/26; A61L 27/12; A61L 27/18

(52) U.S. Cl. CPC A61L 27/20; A61L 27/12; A61L 27/18; A61L 27/025; A61F 2/26; A61B 17/3468; A61L 2400/06

(57) ABSTRACT

A method for augmenting the glans penis provides a minimally invasive introduction of injectable biocompatible material, including hyaluronic acid, to increase the girth of the glans penis while maintaining sensation in the penis. The method initially performs preoperative penile inspection of the girth of a penis. A biocompatible material is injected at a 12 o'clock, a 3 o'clock and 9 o'clock injection point relative to the glans penis. Where damage to the dorsal pedicle may occur, the injection points is at a 10'oclock and a 2 o'clock injection point. The biocompatible material is nontoxic and nonimmunogenic. The biocompatible can also be injected below the corona of the glans penis to create a mushroom effect causing nodules and pearls and intradermal injection causes blebs. Then, postoperative compressive dressing is applied to the penis. Then the penis is measured for postoperative changes in penile girth, and postoperative sexual satisfaction is recorded.

### CROSS REFERENCE OF RELATED APPLICATIONS

This application claims the benefits of U.S. provisional application No. 62171374, filed Jun. 5, 2015, and entitled METHOD OF AUGMENTING A GLANS PENIS WITH INTRADERMAL FILLER MATERIAL INCLUDING HYALURONI ACID GEL, which provisional application is incorporated by reference herein in its entirety.

### FIELD OF THE INVENTION

The present invention relates generally to a minimally invasive penile augmentation method that uses an intradermal filler material, including hyaluronic acid, calcium hydroxyapatite, poly-lactic acid, silicone, and other biocompatible materials. More so, a method for augmenting the glans penis with biocompatible material provides a minimally invasive introduction of injectable biocompatible material, including hyaluronic acid, in augmentation of the glans penis in vivo to increase the girth of the glans penis while still maintaining sensation in the glans penis; and more so, the specific site and level of injection to create bulk and textural stimulation of Graefenburg spot and/or prostate during intercourse.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Typically, penile augmentation consists of various methods and devices that attempts to increase the girth, length, or erectile rigidity of the human penis. Such penile augmentation may be desired for medical reasons, for example, if a patient is unable to penetrate during coitus due to an unusually small penis size; for cosmetic reasons; or to improve a person's self-esteem. Penile augmentation methods and devices can include massaging and stretching the penis, engorging the penis in a vacuum, filling the glans penis with a biocompatible materials, enlargement pills, patches, and ointments.

One example of a penile augmentation device is a penis pump. The penis pump is often a cylinder that is fitted over the penis, with a manual or motorized pump to create a partial vacuum around the penis. The cylinder engorges the penis as blood is drawn in. As vacuum increases in the cylinder, the pressure within the blood vessels of the penis increases as well. However with the penis pump, excessive vacuum pressures may cause vascular damage to the penis.

A known method for penile augmentation involves jelqing, or milking the penis. Jelqing is a physical-therapy technique, intended to achieve a more natural penis enlargement by increasing blood pressure and circulation. Jelqing is performed by repeatedly stroking the penis in a squeezing motion from the base of the shaft to the corona of the glans. There are also mechanisms and instruments that can achieve this same effect.

Yet another method of penile augmentation involves injection of a filler into the glans penis. Generally, the filler is an implant device for expanding the girth and length of a penis. Often, a soft, flexible body is implanted between the shaft and the skin of the penis. The body takes the shape of a partial cylindrical sleeve that has an outer, relatively elastic sheet member and an inner, relatively inelastic sheet member. When implanted, the body covers the corpus cavernosum of the penis and does not or only partially covers the urethra, and extends in length between the glans penis and the base of the penis.

Another penile augmentation method is dermofat graft augmentation phalloplasty. In this method, the phallus is simultaneously increased in length and girth by grafting the dermofat tissue. In addition to this method, methods of grafting a commercially available dermal tissue, which has been obtained by processing homogeneous or heterogeneous skin tissue, in the subcutis of the phallus. Unfortunately, when the hyaluronic acid is not uniformly injected under the epithelium of the glans, the surface of the glans becomes rough and untidy, and even when it is uniformly injected under the epithelium, the surface becomes thin and shiny and thus the skin becomes unnatural. In addition, and the hyaluronic acid is rapidly absorbed such that the expansion effect is reduced and the surface of the glans becomes rough, thereby causing an unnatural shape of the glans.

Other proposals have involved penile augmentation methods and devices. The problem with these penile augmentation methods and devices is that they do not increase the girth of the glans penis while still maintaining sensation in the glans penis; and specifically creating bulk and textural stimulation of Graefenburg spot and/or prostate during intercourse. Even though the above cited gripping devices meets some of the needs of the market, a minimally invasive penile augmentation method that uses an intradermal filler material, including hyaluronic acid, calcium hydroxyapatite, poly-lactic acid, silicone, and other biocompatible materials that are injected at a specific 12 o'clock injection point and a 3 o'clock and 9 o'clock injection point relative to the penis for enhanced girth is still desired.

### SUMMARY OF THE INVENTION

Illustrative embodiments of the disclosure are generally directed to a minimally invasive method for augmenting a penis with intradermal filler material that can increase the girth of the glans penis while still maintaining sensation in the glans penis. Additionally, the specific site and level of injection to create bulk and textural stimulation of Graefenburg spot and/or prostate during intercourse. Additionally, the method can safely and naturally expand the glans without causing problems such as an unnatural rough or shiny surface of the glans and the severe side effects of epidermal necrosis, which are caused by the conventional method of augmenting the glans. This method keeps the natural shape of the glans penis without deforming the surface of the glans penis even when the glans penis augmentation effect is reduced due to dissolution and absorption of the components of the filler.

In one embodiment, the method provides a minimally invasive introduction of injectable biocompatible material, including hyaluronic acid, increases the girth of the glans penis while maintaining sensation in the penis. In operation, the method involves measured injection of the biocompatible material at a 12 o'clock injection point and a 3 o'clock and 9 o'clock injection point relative to the vantage point of looking at a forward, head on view of the penis. In another embodiment, the biocompatible may be injected below the corona of the glans penis to create a mushroom effect. This is advantageous over dermal injection, which often causes nodules and pearls and intradermal injection causes blebs. Further, the biocompatible material that is used to fill the glans penis is nontoxic and nonimmunogenic.

In some embodiments, the steps of the method include at least one of the following: inspecting preoperative penile girth of a penis; injecting biocompatible material into a fascial layer of a glans penis; avoiding dorsal pedicle injury through measured injection of the biocompatible material; providing bulk injection to create dermal nodules and bleb; applying postoperative compressive dressing to penis; measuring postoperative changes in penile girth; and recording postoperative sexual satisfaction.

One objective of the present invention is to increase the girth of the glans penis while still maintaining sensation in the penis as a whole; and specifically creating bulk and textural stimulation of Graefenburg spot and/or prostate during intercourse.

Another objective is to provide a nontoxic and nonimmunogenic filler for injection into the glans penis that substantially increases the girth of the glans penis.

Another objective is to enhance sexual gratification for the patient and his partner through focal increased penile girth.

Another objective is to apply measured injection of the biocompatible material at the 12'oclock, 3 o'clock, and 9 o'clock injection points relative to the glans penis.

Another objective is to avoid dorsal pedicle injury through measured injection of the biocompatible material at the 10'oclock and 2 o'clock injection points relative to the glans penis.

Another objective is to expand the glans penis without causing problems such as an unnatural rough or shiny surface of the glans penis and the severe side effects of epidermal necrosis, which are caused by the conventional method of augmenting the glans penis.

Another objective is to provide increased tissue longevity and low complication rate through the use of biocompatible material as a filler.

Another objective is to provide a simple procedure for penile augmentation.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims and drawings.

### BRIEF DESCRIPTION OF THE DRAWINGS

This invention will now be described, by way of example, with reference to the accompanying drawings, in which:

FIG. 1 illustrates a perspective view of exemplary instruments for performing a method for augmenting a glans penis with a biocompatible material, showing exemplary cannulas of various gauges, in accordance with an embodiment of the present invention;

FIG. 2 illustrates a cross-sectional view of an anatomical diagram of an exemplary penis being injected with biocompatible material, in accordance with an embodiment of the present invention;

FIGS. 3A, 3B, and 3C illustrates perspective views of the method for augmenting the glans penis, where FIG. 3A illustrates a preoperational penis, FIG. 3B illustrates injection at the 10'oclock injection point, FIG. 3C illustrates homogenizing the penis with a roller and a post operational penis, in accordance with an embodiment of the present invention;

FIG. 4 illustrates a side view of an exemplary penis to form a mushrooming effect, in accordance with an embodiment of the present invention; and

FIG. 5 illustrates a flowchart diagram of an exemplary method for augmenting a glans penis with a biocompatible material, such as hyaluronic acid gel, in accordance with an embodiment of the present invention.

Like reference numerals refer to like parts throughout the various views of the drawings.

### DETAILED DESCRIPTION OF THE INVENTION

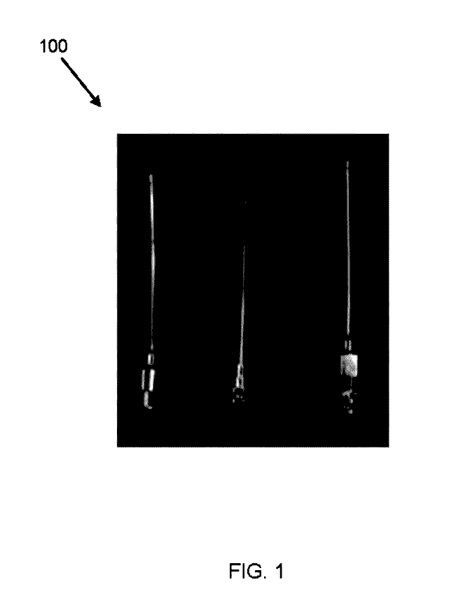

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “first,” “second,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

At the outset, it should be clearly understood that like reference numerals are intended to identify the same structural elements, portions, or surfaces consistently throughout the several drawing figures, as may be further described or explained by the entire written specification of which this detailed description is an integral part. The drawings are intended to be read together with the specification and are to be construed as a portion of the entire “written description” of this invention as required by 35 U.S.C. §112.

In one embodiment of the present invention presented in FIGS. 1-5, a method 200 for augmenting a glans penis 106 uses an intradermal filler material, including hyaluronic acid, calcium hydroxyapatite, poly-lactic acid, silicone, and other biocompatible materials. The method 200 provides a minimally invasive introduction of injectable biocompatible material, including hyaluronic acid, in augmentation of the glans penis 106 in vivo to increase the girth of the glans penis 106 while still maintaining sensation in the glans penis 106. Additionally, the specific site and level of injection to create bulk and textural stimulation of Graefenburg spot and/or prostate during intercourse.

In one embodiment, the method 200 includes at least one of the following steps: inspecting preoperative penile girth of a penis; injecting a biocompatible material into a fascial layer of a glans penis; avoiding dorsal pedicle injury through measured injection of the biocompatible material; providing bulk injection to create dermal nodules and blebs; applying postoperative compressive dressing to the penis; measuring postoperative changes in penile girth; and recording postoperative sexual satisfaction by patient.

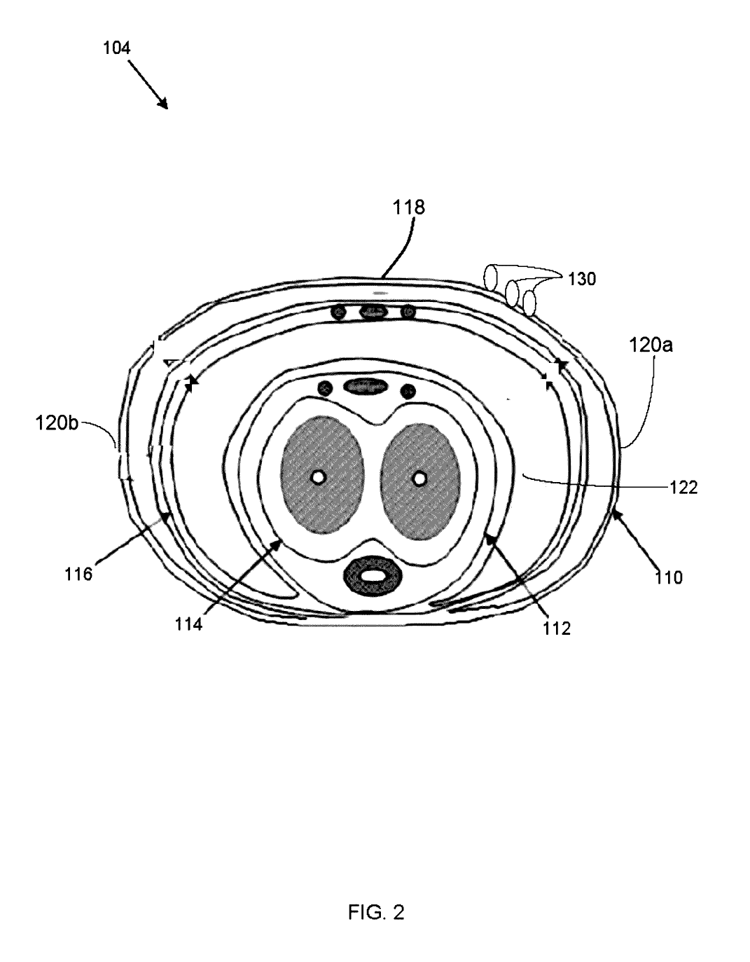

FIG. 1 illustrates a perspective view of an injection instrument, or cannula 100, for injecting the glans penis 106 with a biocompatible material. Thus in one embodiment, the method 200 may be implemented through measured injection of the biocompatible material 108 at a 12 o'clock injection point 118, a 3 o'clock injection point, and a 9 o'clock injection point 120a, 120b relative to the vantage point of looking at a forward, head on view of the glans penis 106. This view, and the specific region of these injection points 118, 120a, 120b are illustrated in FIG. 2.

Though in alternative injection embodiments, where damage to the dorsal pedicle may occur, the injection points may be altered to penetrate approximately at a 10'oclock injection point 126 and a 2 o'clock injection point (not shown) relative to the glans penis 106.

In one alternative embodiment, the biocompatible material 108 may also be injected below the corona of the glans penis 106 to create a mushroom effect comprising dermal nodules 128, microbeads, blebs 130, or superficially at the shaft or glans of the penis 106. Once injected, the biocompatible material 108 is effective in that it is generally nontoxic and nonimmunogenic, as well as not easily adsorbed into the penis tissue.

Due to the biocompatible material 108, the girth of the shaft for the glans penis 106 is safely and naturally expanded without causing problems such as an unnatural rough or shiny surface of the glans penis 106 or the severe side effects of epidermal necrosis, which are caused by the conventional methods of augmenting the glans penis 106. Furthermore, the biocompatible material 108 that is injected into the glans penis 106 is generally nontoxic and nonimmunogenic. Those skilled in the art will recognize that augmentation of the glans penis 106 may be indicated for cosmetic reasons, lack of glans tumescence following implantation of a penile prosthesis, or asymmetry following girth augmentation of the shaft. Many augmentation techniques have been offered to increase the length and girth of a penile shaft, but not the penis 106, especially with biocompatible material 108 injection for strategic placement as nodules 128 or blebs 130.

Those skilled in the art, in light of the present teachings, will recognize that in the last decade, biocompatible material 108 has been shown to possess many properties that suggest its value in several medical applications, particularly in ophthalmology, orthopedics, and aesthetic soft-tissue augmentation with proven efficacy and safety. For the present method 200, the biocompatible material 108 composition, such as a dermal filler, may include, without limitation, hyaluronic acid, calcium hydroxyapatite, poly-lactic acid, silicone, polymethylmethacrylate (PMMA), cross-linked dextran, hydroxypropyl methylcellulose (HPMC), physiological saline, and distilled water.

In some embodiments, the biocompatible material 108 composition may help to rapidly restore volume in the region of the injection points 118, 120a, 120b. Further the biocompatible material 108 does not require pre-testing, such as allergic skin testing, because it does not cause severe allergic reactions, is cheap, and is not easily degraded or absorbed in the body, thus ensuring a long-lasting volume augmentation effect.

Those skilled in the art will also recognize that a major advantage of biocompatible material 108 over nonpermanent fillers, such as fat and collagen, is the increased tissue longevity and low complication rate. The slow degradation of hyaluronic acid gel through cross-linkage enables the several hundred folds longevity of implants compared with the natural polymer, without decreased biocompatibility.

As shown in FIG. 1 the instruments used for augmentation of the glans penis 106 may include a 19, 21, or 23 sized cannula 100. The cannula 100 enables easy injection into the glans penis 106 and proximal area thereof. The cannula 100 may include a tube that can be inserted into the penis 104, often for delivery of the biocompatible material 108. In one possible embodiment, the cannula 100 can surround the inner or outer surfaces of a trocar needle thus extending needle approach to the penis 104 by half or more of the length of the introducer. Pressure may be applied uniformly to flatten focal lumps of the biocompatible material 108 along the glans penis 106.

FIG. 2 shows the anatomic diagrams showing planes of injection into the penis 104. The biocompatible material 108 may be injected in the subcutaneous layer, just “deep to” the dartos fascia 116 just below the coronary sulcus and just above the penopubic junction and the Buck's fascia 112. However, as shown in FIG. 2, the tunica albuginea 114 is never penetrated, as the injection is generally close to the outer skin 110 of the penis 104.

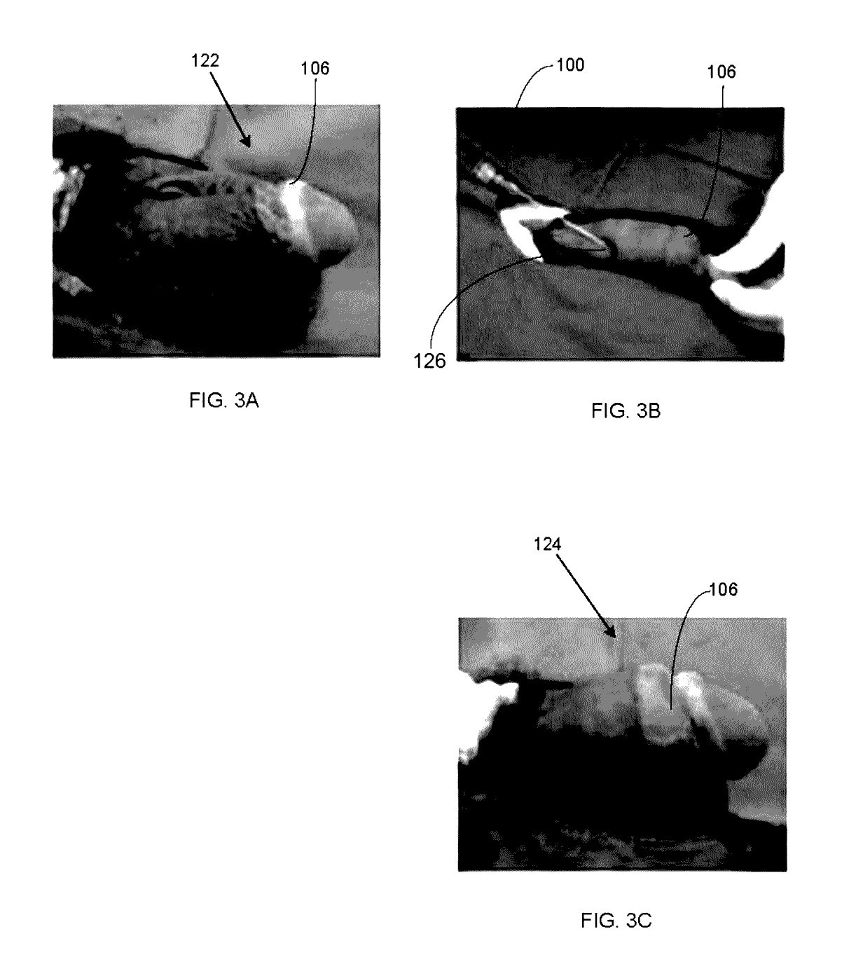

In one alternative embodiment, for the ease of injection into the whole length of the penis 104, and to avoid dorsal pedicle injury, the cannula 100 may be inserted just above penopubic junction or just below the coronal sulcus at a 10 o'clock injection point 126 and a 2 o'clock injection point relative to a front, head on view of the glans penis 106 (FIG. 3B). These alternative injection points 126 may help to avoid dorsal pedicle injury. In a long penis 104 or when there is insufficient enhancement of proximal part of penis 104, additional injection may be performed at just below penopubic junction towards the distal penis 104 bilaterally. To avoid urethral injury, a ventral side was not enhanced directly.

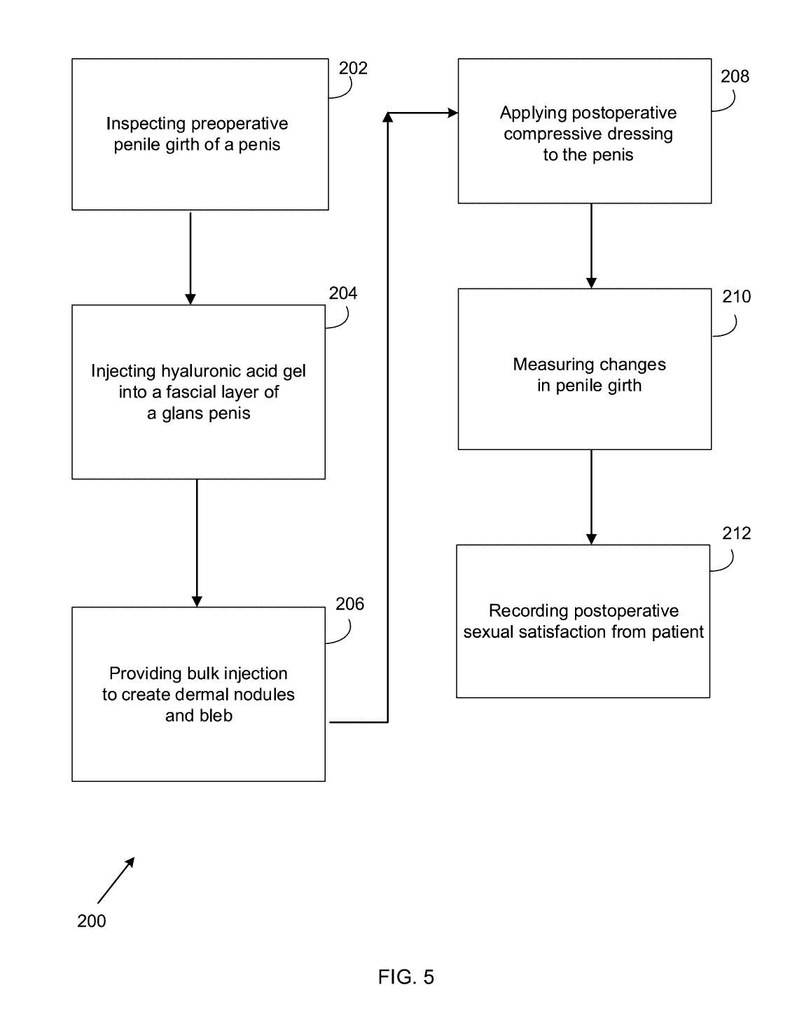

FIG. 5 is a flowchart diagram of the method 200 for augmenting the glans penis 106 with biocompatible material 108. The method 200 may include an initial Step 202 of inspecting preoperative penile girth of a penis 104. FIG. 3A illustrates a preoperative penis 122 having a size and dimension of normal penile girth before augmentation. The glans penis 106 region of the preoperative penis 122 is not swollen or beaded. Any ruler or measuring instrumentation known in the art may be used to measure the girth and length of the penis 104.

A Step 204 may include injecting biocompatible material 108 into a fascial layer of a glans penis 106. In one embodiment, the method 200 may be implemented through measured injection of the biocompatible material 108 at approximately a 12 o'clock injection point 118, a 3 o'clock injection point 120a, and a 9 o'clock injection point 120b relative to the vantage point of looking at a forward, head on view of the glans penis 106. This view, and the specific region of these injection points 118, 120a, 120b are illustrated in FIG. 2. Though in some embodiments, it is not necessary to inject the biocompatible material 108 at all of these injection points 118, 120a, 120b; whereby any combination of injection points 118, 120a, 120b may be injected with the biocompatible material 108.

Looking at FIG. 3B, the biocompatible material 108 is injected into the fascial layer of penile body using a 21G cannula 100 with a “Back and Forth Technique” known in the art. However, in other embodiments, other sizes of cannula 100 may be used, depending on the size of the penis 104. The cannula 100 is generally used for injecting directly into the fascial layer of the glans penis 106.

In some embodiments, it may be necessary to avoid dorsal pedicle injury through measured injection of the biocompatible material 108 at alternative injection points. For the ease of injection into the whole length of the penis 104, the cannula 100 in FIG. 3B is inserted in an alternative injection point, just above penopubic junction or just below the coronal sulcus at 10 o'clock injection point 126 and/or a 2 o'clock injection point of the glans penis 106.

These alternative injection points 126 may be useful for avoiding dorsal pedicle injury. It is significant to note that in a long penis 104 or in circumstances where there is insufficient enhancement of proximal part of penis 104, additional injection may be performed at just below the penopubic junction towards the distal penis 104 bilaterally. Furthermore, to avoid urethral injury, the ventral side may not be injected directly.

In some embodiments, a Step 206 may include bulk injection to create dermal nodules 128 and blebs 130. The biocompatible material 108 may have a tendency to congeal in one region of the penis. Thus forcible homogenizing may be necessary. For example, initial uneven distribution of the biocompatible material 108. The roller 102 may be used can be manipulated with roller 102. After injection of enough amounts, the penis 104 may be stretched and homogenized with the roller 102 for even distribution and to flatten focal lump.

A Step 208 comprises applying postoperative compressive dressing to the penis 104. Postoperative compressive dressing with elastic bandage may be applied and oral antibiotics prescribed for the first week post operation. Additional nighttime compressive dressing with elastic bandage may be recommended to prevent the focal depression from dependent position of the penis 104. Finally, sexual intercourse may be avoided for 1-2 weeks after augmentation of the penis 104.

A Step 210 includes measuring a postoperative penis 124 after the enhanced growth in penile girth after injection of the biocompatible material 108. FIG. 3C illustrates the postoperative penis 124 with increased penile girth immediately postoperative. Penile girth measurement was performed with the penis 104 on full stretch. Changes of penile girth at midshaft were measured by tapeline at 1 month for early results and were followed at 18 months for long-term results. Every measurement of the circumference was performed by one doctor to exclude interpersonal variation.

A final Step 212 includes recording postoperative sexual satisfaction by patient. Patient's subjective visual estimation of penile girth was requested to assess the residual volume of biocompatible material 108 at 1 month and at 18 month. The patients estimated using the visual analogue scale from Gr 1, less than 25% of initial volume; Gr 2, less than 50%; Gr 3, less than 75%; and Gr 4, more than 75% or nearly same as initial volume.

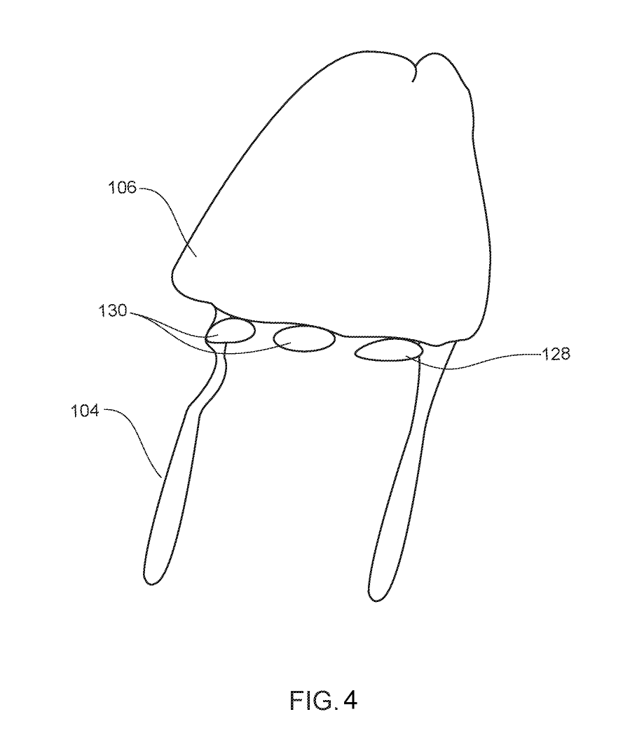

In one alternative embodiment illustrated in FIG. 4, an alternative method of augmenting the glans penis 106 includes injecting collagen below the corona of the glans penis 106. This creates a series of spaced-apart collagen pearls 128 proximal to the glans penis 106. The collagen pearls 128 form a unique mushrooming effect directly below the glans penis 106. Consequently, this alternative method generally increases the diameter of the glans penis 106 and heightens sexual sensations for the participant and his sexual partner. This alternative method 200 may inject a monophasic polydensified hyaluronan acid gel for superficial injection into the glans penis 106. Local anesthetic may be applied, and three injection patterns are possible.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What I claim is:

1. A method for augmenting the glans penis with biocompatible material, the method comprising:
inspecting preoperative penile girth of a penis;
injecting a biocompatible material into a fascial layer of a glans penis;
providing bulk injection to create dermal nodules and blebs;
applying postoperative compressive dressing to the penis;
measuring postoperative changes in penile girth; and
recording postoperative sexual satisfaction by patient.

2. The method of claim 1, wherein the step of injecting a biocompatible material into a fascial layer of a glans penis is performed with a needle or a cannula.

3. The method of claim 1, wherein the step of injecting a biocompatible material into a fascial layer of a glans penis, further includes performing a measured injection of the biocompatible material approximately at a 12 o'clock injection point, a 3 o'clock injection point, and a 9 o'clock injection point of the glans penis.

4. The method of claim 1, wherein the step of injecting a biocompatible material into a fascial layer of a glans penis, further includes performing a measured injection of the biocompatible material at a 12 o'clock injection point and a 3 o'clock injection point of the glans penis.

5. The method of claim 1, wherein the step of injecting a biocompatible material into a fascial layer of a glans penis, further includes performing a measured injection of the biocompatible material at a 12 o'clock injection point and a 9 o'clock injection point of the glans penis.

6. The method of claim 1, wherein the step of injecting a biocompatible material into a fascial layer of a glans penis, further includes performing a measured injection of the biocompatible material at a 3 o'clock injection point and a 9 o'clock injection point of the glans penis.

7. The method of claim 1, further comprising the step of inserting a graft into a fascial layer of the glans penis.

8. The method of claim 1, wherein the step of providing bulk injection to create dermal nodules and blebs comprises pearls and dermal fillers.

9. The method of claim 1, wherein the biocompatible material includes at least one member selected from the group consisting of: hyaluronic acid, calcium hydroxyapatite, poly-lactic acid, silicone, polymethylmethacrylate, cross-linked dextran, hydroxypropyl methylcellulose, physiological saline, and distilled water.

10. A method for augmenting the glans penis with biocompatible material, the method comprising:
inspecting preoperative penile girth of a penis;
injecting a biocompatible material into a fascial layer of a glans penis approximately at a 12 o'clock injection point, a 3 o'clock injection point, and a 9 o'clock injection point on a glans penis;
providing bulk injection to create dermal nodules and blebs;
applying postoperative compressive dressing to the penis;
inserting a graft into a fascial layer of the glans penis;
measuring postoperative changes in penile girth; and
recording postoperative sexual satisfaction by patient.

11. The method of claim 10, wherein the step of injecting a biocompatible material into a fascial layer of a glans penis, further includes performing a measured injection of the biocompatible material at a 12 o'clock injection point, a 3 o'clock injection point, and a 9 o'clock injection point of the glans penis.

12. The method of claim 10, wherein the step of providing bulk injection to create dermal nodules and blebs comprises pearls and dermal fillers.

13. The method of claim 10, wherein the biocompatible material includes at least one member selected from the group consisting of: hyaluronic acid, calcium hydroxyapatite, poly-lactic acid, silicone, polymethylmethacrylate, cross-linked dextran, hydroxypropyl methylcellulose, physiological saline, and distilled water.

14. A method for augmenting the glans penis to create a mushrooming effect proximal to the glans penis, the method comprising:
inspecting preoperative penile girth of a penis;
injecting a biocompatible material below the corona of a glans penis;
creating a mushrooming effect around the glans penis;
providing bulk injection to create dermal nodules and blebs;
applying postoperative compressive dressing to the penis;
measuring postoperative changes in penile girth; and
recording postoperative sexual satisfaction by patient.

15. The method of claim 14, wherein the step of injecting a biocompatible material below the corona of a glans penis is performed with a needle or a cannula.

16. The method of claim 14, wherein the mushrooming effect comprises collagen pearls, microbeads, and blebs that form below the corona of the glans penis.

17. The method of claim 14, wherein the step of injecting a biocompatible material below the corona of a glans penis, further includes performing a measured injection of the biocompatible material at a 10 o'clock injection point and a 2 o'clock injection point of the glans penis.

18. The method of claim 14, wherein the biocompatible material includes at least one member selected from the group consisting of: hyaluronic acid, calcium hydroxyapatite, poly-lactic acid, silicone, polymethylmethacrylate, cross-linked dextran, hydroxypropyl methylcellulose, physiological saline, and distilled water.

19. The method of claim 14, further comprising the step of inserting a graft into a fascial layer of the glans penis.

20. The method of claim 14, wherein the step of providing bulk injection to create dermal nodules and blebs comprises pearls and dermal fillers.
